(19) United States
(12) Patent Application Publication
(10) Pub. No.: US 2015/0139913 A1
(43) Pub. Date: May 21, 2015

(54) MEDICAL USE OF PARTICLES OF TITANIUM AND/OR TITANIUM OXIDE

(71) Applicants: Lars-Magnus Bjursten, (US); Sven-Erik Nilsson, (US); Migrata U.K. Limited, Limassol (CY)

(72) Inventors: Lars-Magnus Bjursten, Limhamn (SE); Sven-Erik Nilsson, Skaret (SE)

(73) Assignee: Migrata U.K. Limited, Limassol (CY)

(21) Appl. No.: 14/405,489

(22) PCT Filed: May 29, 2013
(86) PCT No.: PCT/SE13/50620

§ 371 Date: Dec. 4, 2014

Related U.S. Application Data
(60) Provisional application No. 61/655,093, filed on Jun. 4, 2012.

(30) Foreign Application Priority Data
Jun. 4, 2012 (SE) 1250582-2

(51) Int. Cl. A61K 49/04

(52) U.S. Cl. CPC A61K 49/0419
USPC 424/9.42

(57) ABSTRACT

The present invention describes particles of titanium, titanium alloy, at least one type of titanium oxide or a combination thereof, wherein at least a substantial amount of the particles are of micrometer-millimeter size and are non-spherical, for use as an X-ray contrast agent. The irregular shapes and/or internal pores and cavities of said particles lead to a prolonged retention time in the gastrointestinal tract leading to a longer time period available for X-ray examination. Further, the particles are not only chemically inert, they also exhibit an anti-inflammatory and anti-bacterial effect on the surrounding tissue.

### FIELD OF THE INVENTION

The present invention relates to medical use of particles of titanium, titanium alloy, at least one type of titanium oxide or a combination thereof.

### TECHNICAL BACKGROUND

It has long been known that titanium, its oxides and alloys are biocompatible and hence are used in various medical applications. For example, WO00/64504 discloses a biocompatible, plastic or essentially non-elastic, porous body, such as a grain, with continuous porosity, the openings of cavities and the passages interconnecting them having a width of >about 50 μm for bone tissue. The term “continuous” is said to mean a porosity which allows bone tissue to grow through the porous body. The porous body may be of titanium. The grains may be used for providing ingrowth and growth of connective tissue as well as growth of other cell types leading to cluster of cells, tissues and parts of organs.

In WO 2008/103082 particles of microstructure comprising titanium, titanium alloy, at least one titanium oxide or a combination thereof and their use in some medical applications are described. The disclosed particles have a surface with at least a substantial part consisting of at least one type of titanium oxide. The particles are brought into contact with at least one infected site in a human or animal body by insertion, injection or implantation. The infected site exhibits the inflammatory and/or bacterial condition. Furthermore, WO 2008/103082 refers to an injectable suspension comprising the particles and a fluid vehicle for use as a medicament. Examples of conditions being treated with the injectable suspension are periodontitis, periimplantitis, and osteitis. Due to the fact of the small size of the particles, these could easily be brought into contact with an infected site present in the human or animal body. Specific examples are infected sites in the mouth or dose to the teeth, that is for dental applications, but also e.g. in the intestine or other organs or tissues. An important example is bone tissue, In addition to being injected into inflamed and/or infected tissue, the particles of micro-structure or the injectable suspension disclosed in WO 2008/103082 may also be injected into or inserted into non-inflamed and/or non-infected sites of a human or animal body, e.g. the intestine, liver, spleen, pancreas or the kidneys. One example of use of the particles of microstructure or the injectable suspension are as carriers of medicaments to specific parts of the human or animal body, where the particles either work just as a carriers or as active medicaments in combination with the other medicaments at the site intended to be contacted.

One aim of the present invention is to provide a novel medical use for particles of titanium, titanium alloy, at least one type of titanium oxide or a combination thereof.

### SUMMARY OF THE INVENTION

The stated purpose above is achieved by the present invention providing particles of titanium, titanium alloy, at least one type of titanium oxide or a combination thereof, wherein at least a substantial amount of the particles are of micrometer-millimeter size and are non-spherical, for use as an X-ray contrast agent.

According to the present invention, the particles are of micrometer-millimeter size. In relation to the present invention, this implies that the particles have a “diameter” in the range of 10 μm-5 mm, such as in the range of 10 μm-2 mm, where most of them have a diameter in the range of 10 μm-0.5 mm. Furthermore, the particles according to the present invention are not perfect spheres. Therefore, according to one specific embodiment of the present invention, being non-spherical implies that they have internal pores or cavities or have an irregular shape. The pores may be so called continuous pores going through the particles from one side to the other side, implying at least two openings on the surface of the particle. The pores may also resemble caves with only one opening on the particle surface. These caves may also be pores going deep inside of the particle but not through the entire structure. These caves may be of different length, stretching from one side of the particle to the other side of the same particle or appear as pits on the surface of the particles. The cavities may have an irregular shape and be that of a channel or hole inside the particle. Furthermore, there may also be provided cavities which are nearer the surface and not as deep.

Moreover, the entire structure may have an irregular shape implying that the surface is wavy or also having a geometrical shape not being a sphere, such as having an elongated or asymmetrical cross section or the like. Structures incorporating all of the above features, such being almost oval, having continuous pores and an irregular surface and so on, are of course totally possible according to the present invention.

In contrast to the present invention, EP1146072 discloses spherical particles having a particle size of 5 to 10,000 nm, containing 0.1 to 99.9% by weight of a first oxidic compound, selected from for example titanium and silicon and at least one further oxide of the lanthanides. The particles either have an onion-skin-like structure comprising layers of the different metal oxides or have a homogenous distribution of the metal oxides throughout the particle. Alternatively, one or more metal oxides are embedded in a matrix of one or more metal oxides. Since the lanthanides are paramagnetic, the particles are suitable as an MRI contrast agent. In addition, the particles may also be used as X-ray contrast agents.

In contrast to the particles disclosed in EP1146072, which are spherical, the particles of the present invention have irregular shapes, such as they are in the shape of spheres, spikes, flakes, chips or similar or combinations thereof. In addition, the particles used according to the present invention have an irregular surface and/or internal pores and cavities. As described above, this will lead to a prolonged retention time in the gastrointestinal tract leading to a longer time period available for X-ray examination.

Conventional X-ray contrast agents are often based on barium or iodine. Barium sulphate is used as contrast agent for the gastrointestinal tract and is administered per os or per rectum. A problem associated with the use of barium sulphate as an X-ray contrast agent is the risk of side effects, such as allergic reactions, urticaria and indigestion, such as constipation or diarrhoea.

In contrast to barium sulphate, the particles according to the present invention are chemically inert, and are most unlikely to cause any severe adverse effects when administered per os or per rectum. Furthermore, the particles of the present invention are not only chemically inert, they also exhibit an anti-inflammatory and anti-bacterial effect on surrounding tissue.

In U.S. Pat. No. 4,020,152 there is disclosed another type of contrast agent, namely a radiological contrast product comprising a non-toxic radiopaque salt selected from the group consisting of barium titanate and barium zirconate. The contrast agent disclosed is different from the present invention in many aspects. The first one is the actual material. The second aspect is the shape, both in terms of the actual geometrical shape and size, which are clearly stated features according to the present invention. Moreover, the above mentioned advantages of the particles according to the present invention, such as being chemically inert and also exhibiting an anti-inflammatory and anti-bacterial effect on surrounding tissue, are not addressed or achieved by the material disclosed in U.S. Pat. No. 4,020,152.

Moreover, the particles according to the present invention exhibit a relatively long retention time in the gastrointestinal tract. This is due to the irregular shape of the particles according to the present invention, leading the particles hitching to each other creating larger complexes which will have a prolonged retention time compared to an individual particle or to spherical particles of approximately the same size.

Specific embodiments according to the present invention will be described in more detail below. These embodiments should be regarded just as such specific ones, and should not be interpreted as a limitation of the present invention. The scope of the invention is defined by the appended claims.

### SPECIFIC EMBODIMENTS OF THE INVENTION

The material of the particles is essential in relation to the present invention. Firstly, it is the matter of the possible compositions of the particles, where titanium is an element always being present. However, it is important to understand that the base metal titanium can be present in a particle according to the present invention as an alloy, as pure metal titanium, that is with only possible small amounts of impurities, as a titanium oxide, or a combination thereof. The possible small amounts of impurities in pure titanium are normally oxides or some metals, but could also consist of other chemicals. Moreover, titanium oxide is always present in some extent on the surface of the particles. Different types of possible titanium oxides are titanium dioxide (TiO₂), also known as titanium(IV) oxide or titania, titanium monoxide (TiO), also known as titanium(II) oxide, dititanium trioxide (Ti₂O₃), also known as titanium(III) oxide, Ti₃O and Ti₂O. Examples of titanium alloys include alloys comprising titanium and one or more of aluminium, gallium, germanium, carbon, oxygen, nitrogen, molybdenum, vanadium, tantalum, niobium, manganese, iron, chromium, cobalt, nickel, copper and silicon.

According to one specific embodiment of the present invention, the particles are made of titanium dioxide. Another most valid mixture is a set of grains of titanium dioxide particles and titanium metal particles, and possibly also particles being of metal but having a relatively extensive oxide surface coating.

Secondly, the geometrical structure and size are important. According to the present invention, the particles are non-spherical implying having e.g. irregular shapes, that is they are in the shape of spheres, spikes, flakes, chips or similar or combinations thereof. In addition, the particles used according to the present invention have irregular surfaces, i.e. they have internal pores, e.g. continuous such, or cavities.

According to one specific embodiment of the present invention, the particles used as an X-ray contrast agent are non-spherical and have an average length from one side to the opposite side, through a geometrical centre, of ≦5 mm. According to one specific embodiment of the present invention, the particles have an average length from one side to the opposite side, through a geometrical centre, of in the range of ≦2 mm, more specifically an average length from one side to the opposite side, through a geometrical centre, of ≦1 mm. More specifically, the particles used according to the present invention have an average length from one side to the opposite side, through a geometrical centre, of ≦0.5 mm, even more specifically of ≦0.2 mm and still more specific of ≦0.1 mm. According to one specific embodiment of the present invention, the particles used as an X-ray contrast agent have an average length from one side to the opposite side, through a geometrical centre, in the range of 0.01-0.1 mm, more specifically an average length from one side to the opposite side, through a geometrical centre, of <0.05 mm. It is important to appreciate that the particles used according to the present invention may be present as a mixture of particles having different average length from one side to the opposite side, through a geometrical centre, that is the size distribution of the used particles may be in the range of 0.01 mm-5 mm.

According to one specific embodiment of the present invention, the particles are contained in a medical product in the form of a solution, dispersion, suspension, a tablet, a pastille, a suppository, or any other dosage form intended for oral or rectal administration. The products for oral administration may be consumed for example as a liquid, a tablet for swallowing or a chewing tablet. In order to facilitate the administration of the particles for use according to the present invention, the medical product according to the present invention may also comprise at least one functional additive, which may be directed to affecting the physical properties of the medical product. Examples are additives like dispersants, emulsifiers or gelatinizing agents, or an agent increasing the surface tension, making sure that the product has the correct form or physical properties for a specific administration. Another possibility is to add a dry mixture of a weak acid and a weak base so that the particles are dispersed by the carbon dioxide gas that is released when the mixture comes in contact with the wet environment of the gastrointestinal tract.

Moreover, according to one embodiment of the present invention, the medical product used as an X-ray contrast agent also comprises at least one functional additive directed to affecting the therapeutic properties of the medical product. Examples are antibiotics, anti-inflammatory agents, steroids, NSAID (non steroidal anti-inflammatory drug), drugs to treat diarrhoea or constipation.

Different functional additives directed to affecting the therapeutic properties of the medical product may be used in the same medical product. For example, antibiotics and anti-inflammatory agents may both be present in the medical product to be used according to the present invention.

According to one embodiment of the present invention, the particles are used as an inert X-ray contrast agent in the gastrointestinal tract of a human or animal. The usage is intended to leave the human or animal gastrointestinal tract unaffected by the use of the particles.

According to one specific embodiment of the present invention, the particles used as an X-ray contrast agent are used for monitoring the therapeutic effect of the particles when administered into the human or animal body. By visualizing the administered particles by X-ray examination, the location of the particles and the status of the surrounding tissue are determined. In this way, the therapeutic effect of administered particles can be monitored. As described above, according to the present invention, the particles can be present in a medical product also comprising a compound having therapeutic effect. Since the particles, according to the present invention, are used as an X-ray contrast agent, the location of the particles in the medical product can be determined by X-ray examination at the same time as the effect of the compound having therapeutic effect on status of the surrounding tissue is examined. The therapy can hence be evaluated with regard to how much of the particles (that is the medical product also comprising a compound) having therapeutic effect actually reach the site intended.

1. Particles of titanium, titanium alloy, at least one type of titanium oxide or a combination thereof, wherein at least a substantial amount of the particles are of micrometer-millimeter size and are non-spherical, for use as an X-ray contrast agent.

2. The particles according to claim 1, wherein non-spherical implies having internal pores or cavities or having an irregular shape.

3. The particles according to claim 1, wherein the particles are contained in a medical product in the form of a solution, dispersion, suspension, a tablet, a pastille, a suppository, or any other dosage form intended for oral or rectal administration.

4. The particles according to claim 3, wherein the medical product also comprises at least one functional additive.

5. The particles according to claim 4, wherein the at least one functional additive is directed to affecting the physical properties of the medical product.

6. The particles according to claim 4, wherein the at least one functional additive is directed to affecting the therapeutic properties of the medical product.

7. The particles according to claim 1, wherein the particles are made of titanium dioxide.

8. The particles according to claim 1, for use as an inert X-ray contrast agent for use in the gastrointestinal tract.

9. The particles according to claim 1, for monitoring the therapeutic effect of said particles when administered into the human or animal body.
